(19) United States
(12) Patent Application Publication
(10) Pub. No.: US 2026/0193157 A1
(43) Pub. Date: Jul. 9, 2026

(54) METHODS FOR FORMING LIGHT OLEFINS WITH CATALYST RECYCLE

(71) Applicant: Dow Global Technologies LLC, Midland, MI (US)

(72) Inventors: Matthew T. Pretz, Lake Jackson, TX (US); Liwei Li, Lake Jackson, TX (US); Yang Yang, Pearland, TX (US); Adrianus Koeken, Terneuzen (NL); Lin LUO, Sugarland, TX (US); Quan Yuan, Lake Jackson, TX (US); Chi-Wei Tsang, Lake Jackson, TX (US)

(73) Assignee: Dow Global Technologies LLC, Midland, MI (US)

(21) Appl. No.: 19/128,409

(22) PCT Filed: Nov. 22, 2023
(86) PCT No.: PCT/US2023/080906

§ 371 Date: May 8, 2025

Related U.S. Application Data
(60) Provisional application No. 63/428,498, filed on Nov. 29, 2022.

(51) Int. Cl. C07C 15/46; B01J 38/20; C07C 11/04; C07C 11/06; C07C 11/08

(52) U.S. Cl. CPC C07C 15/46; B01J 38/20; C07C 11/04; C07C 11/06; C07C 11/08

(57) ABSTRACT

According to embodiments disclosed herein, a method for forming light olefins in a reactor system may include reacting a feed stream in the presence of a catalyst to form a deactivated catalyst, passing the deactivated catalyst to a combustor and processing the deactivated catalyst to produce a reactivated catalyst, combining a portion of the reactivated catalyst with the deactivated catalyst upstream of the combustor to form a mixed catalyst stream and contacting the mixed catalyst stream with a first oxygen-containing gas stream upstream of the combustor, and passing the mixed catalyst stream to the combustor and contacting the mixed catalyst stream with a second oxygen-containing gas stream while in the combustor, where the molar flow rate of the first oxygen-containing gas stream is 1% to 15% of the combined molar flow rate of the first oxygen-containing gas stream and second oxygen-containing gas stream.

### CROSS-REFERENCE TO RELATED APPLICATIONS

This application claims the benefit of U.S. Provisional Application Ser. No. 63/428,498 filed Nov. 29, 2022, the entire disclosure of which is hereby incorporated herein by reference.

### TECHNICAL FIELD

Embodiments described herein generally relate to chemical processing and, more specifically, to methods and systems for light olefin production.

### BACKGROUND

Light olefins, such as propylene, may be used as base materials to produce many different materials, such as polypropylene, isopropanol, and acrylic acid, which may be used in, e.g., packaging, construction, and textiles. As a result of this utility, there is a worldwide demand for light olefins. Suitable processes for producing light olefins generally depend on the given chemical feed and include those that utilize fluidized catalysts. For example, light olefins may be formed by the catalytic dehydrogenation of alkanes in a fluidized bed reactor. However, there is a need for improvement in the systems and associated catalysts used to make light olefins.

### SUMMARY

Conventional processes for forming light olefins may pass a deactivated catalyst to a catalyst processing system comprising a combustion step and a rejuvenation step in order to heat and reactivate the catalyst, but these processes have several deficiencies. For example, deactivated catalyst may directly enter the combustor where a relatively high amount of coke is deposited on the deactivated catalyst so that after the deactivated catalyst is processed, the catalyst still comprises a relatively high amount of coke. In another example, deactivated catalyst may enter the combustor at a relatively low temperature and may not uniformly heat up to a regeneration temperature, thus resulting in some catalyst not achieving proper regeneration. In another example, catalyst with coke may enter the combustor and not be distributed uniformly which will preferentially consume the available oxygen resulting in poor fuel air mixing and insufficient combustion. Additionally, some conventional processes may treat at least a portion of the deactivated catalyst with a gas stream in one or more separate units before entering a combustor, where there will be higher costs associated with having the one or more separate units.

Described herein are processes for forming light olefins which may, in some embodiments, overcome these deficiencies. According to embodiments described herein, a portion of reactivated catalyst may be recycled and combine with deactivated catalyst while upstream of the combustor to form a mixed catalyst stream that then enters the combustor. This combination of the recycled portion of reactivated catalyst with the deactivated catalyst may result in reducing the amount of coke deposited on the deactivated catalyst before entering the combustor, thus exposing more catalyst active sites of the deactivated catalyst and improving combustion of a supplemental fuel in the regenerator. In addition, this combination of the recycled portion of reactivated catalyst with the deactivated catalyst may heat the deactivated catalyst before entering the combustor, thus achieving the target regeneration temperature more quickly and achieving a more uniform regeneration of catalyst. In addition, if the coke is combusted prior to entering the combustor where supplemental fuel is mixed with air, the distribution of the catalyst may be less important because maldistribution may not impact the fuel to air mixture within the combustor to the same degree as otherwise. In one or more embodiments, the combining of the deactivated catalyst and the recycled portion of the reactivated catalyst does not require mixing in one or more separate units, thus lowering the costs associated with this combination step. Further, the deactivated catalyst and the recycled portion of the reactivated catalyst may be contacted with one oxygen-containing gas stream before entering a combustor and a separate oxygen-containing gas stream may be introduced to the combustor, where the oxygen-containing gas stream used to pass the deactivated catalyst and the recycled portion of the reactivated catalyst to the combustor contributes to the total oxygen-containing gas of the combustor and reduces the costs associated with providing the separate oxygen-containing gas stream directly to the combustor.

According to one or more embodiments of the present disclosure, a method for forming light olefins in a reactor system may comprise reacting a feed stream in the presence of a catalyst in a reactor to form a product stream and a deactivated catalyst comprising coke, separating at least a portion of the product stream from the deactivated catalyst, passing the deactivated catalyst to a combustor in a catalyst processing portion of the reactor system and processing the deactivated catalyst to produce a reactivated catalyst and a flue gas, wherein coke is removed from the deactivated catalyst in the combustor, separating the reactivated catalyst from the flue gas and separating the reactivated catalyst into a first portion and a second portion, passing the first portion of the reactivated catalyst to the reactor, combining the second portion of the reactivated catalyst with the deactivated catalyst upstream of the combustor to form a mixed catalyst stream, wherein the mixed catalyst stream is contacted with a first oxygen-containing gas stream upstream of the combustor and wherein coke on the deactivated catalyst is oxidized when contacted with the first oxygen-containing gas stream upstream of the combustor, and passing the mixed catalyst stream to the combustor and contacting the mixed catalyst stream with a second oxygen-containing gas stream while in the combustor, wherein the sum of the molar flow rate of the first oxygen-containing gas stream and the molar flow rate of the second oxygen-containing gas define a total oxygen-containing gas molar flow rate, and the molar flow rate of the first oxygen-containing gas stream is 1% to 15% of the total oxygen-containing gas molar flow rate.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawing and claims, or recognized by practicing the described embodiments. The drawing is included to provide a further understanding of the embodiments and, together with the detailed description, serves to explain the principles and operations of the claimed subject matter. However, the embodiment depicted in the drawing is illustrative and exemplary in nature, and not intended to limit the claimed subject matter.

### BRIEF DESCRIPTION OF THE DRAWINGS

The following detailed description may be better understood when read in conjunction with the following drawings, in which:

FIG. 1 schematically depicts a reactor system, according to one or more embodiments of the present disclosure; and

FIG. 2 schematically depicts a cutaway view of a combustor of a catalyst processing portion of a reactor system, according to one or more embodiments of the present disclosure.

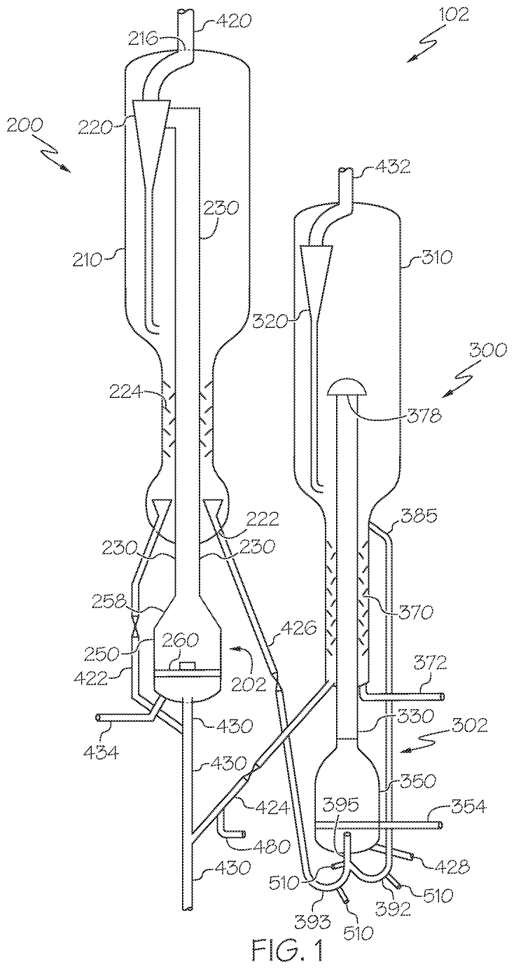

When describing the simplified schematic illustration of FIG. 1, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

Reference will now be made in greater detail to various embodiments, some of which are illustrated in the accompanying drawing.

### DETAILED DESCRIPTION

Embodiments presently disclosed are described in detail herein in the context of the reactor system of FIG. 1 operating as a fluidized dehydrogenation reactor system to produce light olefins. However, it should be understood that the principles disclosed and taught herein may be applicable to other systems which utilize different system components oriented in different ways, or different reaction schemes utilizing various catalyst compositions. For example, the concepts described may be equally applied to other systems with alternate reactor units and regeneration units, such as those that operate under non-fluidized conditions or include downers rather than risers. Additionally, it is contemplated that light olefins may be produced from a variety of hydrocarbon feed streams and by utilizing different reaction mechanisms. For example, light olefins may be catalytically produced by at least dehydrogenation reactions, cracking reactions, dehydration reactions, and methanol-to-olefin reactions. In some embodiments, oxygen carrier materials may also be utilized to selectively combust hydrogen, as is described herein. These reaction types may utilize different feed streams and/or different catalysts to produce light olefins. It should be further understood that not all portions of FIG. 1 should be construed as essential to the claimed subject matter.

Now referring to FIG. 1, an example reactor system 102 that may be suitable for use with the methods and/or apparatuses described herein is schematically depicted. The reactor system 102 generally comprises multiple system components, such as a reactor portion 200 and a catalyst processing portion 300. As described herein, “system components” refer to portions of the reactor system 102, such as reactors, separators, transfer lines, combinations thereof, and the like. As used herein in the context of FIG. 1, the reactor portion 200 generally refers to the portion of a reactor system 102 in which the major process reaction takes place (e.g., dehydrogenation) to form the product stream. A feed stream enters the reactor portion 200, is converted to a product stream (containing product and unreacted feed), and exits the reactor portion 200. The reactor portion 200 comprises a reactor 202 which may include an upstream reactor section 250 and a downstream reactor section 230. According to one or more embodiments, as depicted in FIG. 1, the reactor portion 200 may additionally include a catalyst separation section 210, which serves to separate the catalyst from the chemical products formed in the reactor 202. Also, as used herein, the catalyst processing portion 300 generally refers to the portion of the reactor system 102 where the catalyst is in some way processed, such as by combustion, to, e.g., improve catalytic activity by decoking and/or heat the catalyst. The catalyst processing portion 300 may comprise a combustor 350 and a riser 330, and may additionally comprise a catalyst separation section 310. In one or more embodiments, the catalyst separation section 210 may be in fluid communication with the combustor 350 (e.g., via standpipe 426) and the catalyst separation section 310 may be in fluid communication with the upstream reactor section 250 (e.g., via standpipe 424 and transport riser 430). In one or more embodiments, catalyst from the catalyst separation section 210 (sometimes referred to as deactivated catalyst) is passed towards the combustor 350 via standpipe 426. In one or more embodiments, a portion of the catalyst from the catalyst processing portion 300 (sometimes referred to as a first portion of a reactivated catalyst) may pass to the upstream reactor section 250 via standpipe 424 and transport riser 430 and a portion of the catalyst from the catalyst processing portion 300 (sometimes referred to as a second portion of a reactivated catalyst) may be recycled and pass towards the combustor 350 via standpipe 385.

As is described herein, the deactivated catalyst from standpipe 426 may combine with the second portion of reactivated catalyst from standpipe 385 to form a mixed catalyst stream that then enters the combustor 350. Such a second portion of reactivated catalyst may be considered a recycle stream within the catalyst processing portion 300. Described in detail, hereinbelow, are advantages of such an arrangement.

Generally as is described herein, in embodiments illustrated in FIG. 1, catalyst is cycled between the reactor portion 200 and the catalyst processing portion 300. It should be understood that when “catalysts” are referred to herein, they may refer to solid materials that are catalytically active for a desired reaction, or may equally refer to other particulate solids referenced with respect to the system of FIG. 1 which do not necessarily have catalytic activity but affect the reaction, such as oxygen carriers. The terms “catalytic activity” and “catalyst activity” refer to the degree to which the catalyst is able to catalyze the reactions conducted in the reactor system. The catalyst that exits the reactor portion 200 may be deactivated catalyst. As used herein, “deactivated” may refer to a catalyst which has reduced catalytic activity or is cooler as compared to catalyst entering the reactor portion 200. However, deactivated catalyst may maintain some catalytic activity. Reduced catalytic activity may result from contamination with a substance such as coke. Reactivation (sometimes called “regeneration” herein) may remove the contaminant such as coke, raise the temperature of the catalyst, and/or restructure catalytic sites to recover or improve the dehydrogenation and/or combustion activity of the catalyst. In embodiments, deactivated catalyst may be reactivated by catalyst reactivation in the catalyst processing portion 300. The deactivated catalyst may be reactivated by, but not limited to, removing coke by combustion, recovering catalyst acidity, oxidizing the catalyst, heating the catalyst, other reactivation process, or combinations thereof. In some embodiments, the catalyst may be heated during reactivation by combustion of a supplemental fuel, such as hydrogen, methane, ethane, propane, natural gas, or combinations thereof.

As described with respect to FIG. 1, the feed stream may enter feed inlet 434 into the reactor 202, and the product stream may exit the reactor system 102 via pipe 420. According to one or more embodiments, the reactor system 102 may be operated by feeding a chemical feed (e.g., in a feed stream) and a fluidized catalyst into the upstream reactor section 250. The chemical feed contacts the catalyst in the upstream reactor section 250, and each flow upwardly into and through the downstream reactor section 230 to produce a chemical product.

Now referring to FIG. 1 in detail, the reactor portion 200 may comprise an upstream reactor section 250, a transition section 258, and a downstream reactor section 230, such as a riser. The transition section 258 may connect the upstream reactor section 250 with the downstream reactor section 230. As depicted in FIG. 1, the upstream reactor section 250 may be positioned below the downstream reactor section 230. Such a configuration may be referred to as an upflow configuration in the reactor 202. The upstream reactor section 250 may include a vessel, drum, barrel, vat, or other container suitable for a given chemical reaction. As depicted in FIG. 1, the upstream reactor section 250 may be connected to the downstream reactor section 230 via the transition section 258. The upstream reactor section 250 may generally comprise a greater cross-sectional area than the downstream reactor section 230. The transition section 258 may be tapered from the size of the cross-section of the upstream reactor section 250 to the size of the cross-section of the downstream reactor section 230 such that the transition section 258 projects inwardly from the upstream reactor section 250 to the downstream reactor section 230. For example, the transition section 258 may be a frustum.

The upstream reactor section 250 may be connected to a transport riser 430, which, in operation may provide reactivated catalyst in a feed stream to the reactor portion 200. The reactivated catalyst and/or reactant chemicals may be mixed with a distributor 260 housed in the upstream reactor section 250. The catalyst entering the upstream reactor section 250 via transport riser 430 may be passed through standpipe 424 to a transport riser 430, thus arriving from the catalyst processing portion 300. In some embodiments, catalyst may come directly from the catalyst separation section 210 via standpipe 422 and into a transport riser 430, where it enters the upstream reactor section 250, where in such embodiments some of the catalyst is not passed through the catalyst processing portion 300. The catalyst can also be fed via standpipe 422 directly to the upstream reactor section 250 (not depicted in FIG. 1). This catalyst may be somewhat deactivated, but may still, in some embodiments, be suitable for reaction in the upstream reactor section 250, particularly when used in combination with reactivated catalyst.

Still referring to FIG. 1, in one or more embodiments, based on the shape, size, and other processing conditions (such as temperature and pressure) in the upstream reactor section 250 and the downstream reactor section 230, the upstream reactor section 250 may operate as a fluidized bed, such as in a fast fluidized, turbulent, or bubbling bed upflow reactor, while the downstream reactor section 230 may operate in more of a plug flow manner, such as in a riser reactor. For example, the reactor 202 of FIG. 1 may comprise an upstream reactor section 250 operating as a fast fluidized, turbulent, or bubbling bed reactor and a downstream reactor section 230 operating as a dilute phase riser reactor, with the result that the average catalyst and gas flow moves concurrently upward. As the term is used herein, “average flow” refers to the net flow, i.e., the total upward flow minus the retrograde or reverse flow, as is typical of the behavior of fluidized particles in general. As described herein, a “fast fluidized” reactor may refer to a reactor utilizing a fluidization regime wherein the superficial velocity of the gas phase is greater than the choking velocity and may be semi-dense in operation. As described herein, a “turbulent” reactor may refer to a fluidization regime where the superficial velocity of less than the choking velocity and is more dense than the fast fluidized regime. As described herein, a “bubbling bed” reactor may refer to a fluidization regime wherein well defined bubbles in a highly dense bed are present in two distinct phases. The “choking velocity” refers to the minimum velocity required to maintain solids in the dilute-phase mode in a vertical conveying line. As described herein, a “dilute phase riser” may refer to a riser reactor operating at transport velocity, where the gas and catalyst have about the same velocity in a dilute phase.

According to embodiments, the chemical product and the catalyst may be passed out of the downstream reactor section 230 to a separation device 220 in the catalyst separation section 210, where the catalyst is separated from the chemical product, which is transported out of the catalyst separation section 210. According to one or more embodiments, following separation from vapors in the separation device 220, the catalyst may generally move through the stripper 224 to the catalyst outlet port 222 where the catalyst is transferred out of the reactor portion 200 via standpipe 426 and towards the catalyst processing portion 300.

According to one or more embodiments, the separation device 220 may be a cyclonic separation system, which may include two or more stages of cyclonic separation. In embodiments where the separation device 220 comprises more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the catalyst from the product gas. It should be understood that any primary cyclonic separation device may be used in the presently disclosed embodiments.

Still referring to FIG. 1, the separated deactivated catalyst is passed from the catalyst separation section 210 towards the combustor 350 via standpipe 426 and J-bend 393. The deactivated catalyst is then combined with the second portion of reactivated catalyst passed via standpipe 385 and J-bend-392. The second portion of the reactivated catalyst may be passed from the catalyst separation section 310 and recycled towards the combustor 350 of the catalyst processing portion 300 via standpipe 385 and J-bend 392. The deactivated catalyst combines with the second portion of the reactivated catalyst to form a mixed catalyst stream. The combination of the deactivated catalyst and the second portion of the reactivated catalyst may be done in pipe 395 that may be in fluid communication with combustor 350 such that the components of the mixed catalyst stream may contact the first oxygen-containing gas stream and thoroughly mix for at least 1 second, at least 2 seconds, at least 3 seconds, at least 4 seconds, at least 5 seconds, or even greater, such as about 8 seconds, before the mixed catalyst stream is passed to combustor 350. The mixed catalyst stream may then enter the combustor 350. The mixing of these streams is generally upstream of their insertion into the combustor 350.

In one or more embodiments, the first oxygen-containing gas may contact the mixed catalyst stream via one or more of pipes 510 before the mixed catalyst stream is passed to the combustor 350. The first oxygen-containing gas stream may enter via pipe 510 at J-bend 393, the first oxygen-containing gas stream may enter via pipe 510 at J-bend 392, and/or the first oxygen-containing gas stream may enter via pipe 510 at pipe 395. The first oxygen-containing gas may enter at one or more of the pipes 510. When the first oxygen containing-gas enters via pipe 510 at J-bend 393, the first oxygen-containing gas will contact at least a portion of the deactivated catalyst passing in standpipe 426 and then contact the second portion of the reactivated catalyst that is combined with the deactivated catalyst in pipe 395. Prior to contact with line 510, the catalyst in line 426 may be in the presence of an inert gas such as nitrogen or steam. When the first oxygen containing-gas enters via pipe 510 at J-bend 392, the first oxygen-containing gas will contact at least a portion of the second portion of the reactivated catalyst passing in standpipe 385 before the deactivated catalyst is combined with the second portion of the reactivated catalyst. When the first oxygen containing-gas enters via pipe 510 at pipe 395, the first oxygen containing-gas will contact the mixed catalyst stream in pipe 395 that contains both the deactivated catalyst and the second portion of the reactivated catalyst. The use of oxygen from pipes 510 may additionally contribute to less oxygen needing to be added directly to the combustor 350, which may have advantages in terms of achieving a desired fluidization regime in the combustor 350.

Without being bound by any particular theory, it is believed that combining the deactivated catalyst with the second portion of the reactivated catalyst to form the mixed catalyst stream and contacting the mixed catalyst stream with the first oxygen-containing gas stream reduces the amount of coke deposited on the deactivated catalyst before the mixed catalyst stream enters the combustor 350. Further, it is believed that combining the deactivated catalyst with the second portion of the reactivated catalyst to form the mixed catalyst stream and contacting the mixed catalyst stream with the first oxygen-containing gas stream pre-oxidizes the coke on the deactivated catalyst before the deactivated catalyst enter the combustor 350 and also helps achieve thorough mixing of the deactivated catalyst and the second portion of the reactivated catalyst in pipe 395. Altogether, it is believed that this will result in an increase in the performance of the catalyst in combusting a supplemental fuel in the combustor 350. In additional embodiments, the combining of the catalyst streams may introduce fluidization efficiencies by having all catalyst enter the center of the combustor 350.

Further, without being bound by any particular theory, it is believed that combining the deactivated catalyst with the second portion of the reactivated catalyst to form the mixed catalyst stream and contacting the mixed catalyst stream with the first oxygen-containing gas stream heats the deactivated catalyst prior to entering the combustor 350. In many conventional processes, various distributors, mixing equipment, such as pipes, baffles, attempt to quickly spread and mix the deactivated catalyst in order to heat the catalyst to a target regeneration temperature. However, in these conventional processes, the mixing equipment may not fully mix the catalyst uniformly throughout a catalyst processing portion, or at least to the degree that the presently disclosed pre-mixing scheme may achieve, thus resulting in areas within the catalyst processing portion where the catalyst is not sufficiently heated, thus resulting in some uneven deactivated catalyst regeneration. In the processes described herein, according to one or more embodiments, because the deactivated catalyst is able to be heated prior to entering the combustor 350, the deactivated catalyst can reach the target regeneration temperature more quickly and achieve a more uniform regeneration of the deactivated catalyst. For example, it is believed that a higher conversion of a supplemental fuel, such as methane, may be achieved due to the deactivated catalyst being heated prior to entering the combustor 350 when compared to processes that just directly pass the deactivated catalyst from the reactor portion 200 to the combustor 350.

In one or more embodiments, combining the second portion of the reactivated catalyst with the deactivated catalyst upstream of the combustor to form the mixed catalyst stream is done in pipe 395 in a dense phase lift fluidization regime. The term “dense phase lift fluidization regime” may refer to a fluidization regime that results in the contacting of the mixed catalyst stream with the first oxygen-containing gas stream where the first oxygen-containing gas stream has a velocity that results in thorough contacting of the first oxygen-containing gas stream and the mixed catalyst stream, yet the velocity is not high enough to transport the mixed catalyst stream into combustor 350 before thorough mixing of the mixed catalyst stream is achieved. It is contemplated that pipe 395 may be a pipe or vessel that has various cross-sectional shapes and sizes and has a length that allows the mixed catalyst stream and the first oxygen-containing gas stream to thoroughly mix before the mixed catalyst stream enters combustor 350.

In one or more embodiments, pipe 395 may be operated with a superficial gas velocity of from 0.3 m/s to 5 m/s, such as from 0.4 m/s to 2.5 m/s, from 0.6 m/s to 2.3 m/s from 0.7 m/s to 2.2 m/s, from 0.9 m/s to 2.2 m/s, from 1.0 m/s to 2.1 m/s, or from 1.5 m/s to 2.1 m/s. In one or more embodiments, pipe 395 may be operated with a solid flux of from 245 kg/m²-s to 1710 kg/m²-s, such as from 300 kg/m²-s to 1500 kg/m²-s, from 400 kg/m²-s to 1450 kg/m²-s, from 400 kg/m²-s to 1400 kg/m²-s, from 500 kg/m²-s to 1,350 kg/m²-s, from 500 kg/m²-s to 1,300 kg/m²-s, from 550 kg/m²-s to 1,220 kg/m²-s, or from 600 kg/m²-s to 1,000 kg/m²-s.

In one or more embodiments, combining the second portion of the reactivated catalyst with the deactivated catalyst upstream of the combustor 350 to form the mixed catalyst stream results in a temperature difference between the second portion of the reactivated catalyst and the deactivated catalyst of less than or equal to 10° C. prior to entering the combustor 350. For example, the temperature difference may be less than or equal to 10° C., less than or equal to 9° C., less than or equal to 8° C., less than or equal to 7° C., less than or equal to 6° C., less than or equal to 5° C., less than or equal to 4° C., less than or equal to 3° C., less than or equal to 2° C., or even less than or equal to 1° C.

In one or more embodiments, the mixed catalyst stream enters the catalyst processing portion 300 through a bottom center portion of the combustor 350. The center portion of the combustor 350 refers to the point that is approximately half the diameter relative to any two points along the circumference of the combustor body. It is noted that the mixed catalyst stream does not have to enter the combustor 350 at the exact center point of the bottom of the combustor 350 and that the mixed catalyst stream may enter the combustor 350 at any point along the bottom of the combustor 350 that allows the stream to properly rise upwards towards the riser 330.

In one or more embodiments, the mixed catalyst stream may enter the combustor 350 at a flow rate of from 0.5 m/s to 4.0 m/s. For example, the mixed catalyst stream may enter the combustor 350 at a flow rate of from 0.5 m/s to 3.5 m/s, from 0.5 m/s to 3.0 m/s, from 0.5 m/s to 2.5 m/s, from 0.5 m/s to 2.0 m/s, from 0.5 m/s to 1.5 m/s, from 0.5 m/s to 1.0 m/s, from 1.0 m/s to 4.0 m/s, from 1.5 m/s to 4.0 m/s, from 2.0 m/s to 4.0 m/s, from 2.5 m/s to 4.0 m/s, from 3.0 m/s to 4.0 m/s, from 3.5 m/s to 4.0 m/s, from 1.0 m/s to 3.5 m/s, from 1.5 m/s to 3.0 m/s, or from 2.0 m/s to 3.0 m/s.

The term “oxygen-containing gas” may refer to any gas that comprises at least 0.5% by mass oxygen. For example, oxygen-containing gas may comprise at least 1% by mass, at least 5% by mass, at least 10% by mass, at least 20% by mass, at least 30% by mass, at least 40% by mass, at least 50% by mass, at least 60% by mass, at least 70% by mass, at least 80% by mass, or at least 90% by mass oxygen. In some embodiments, oxygen-containing gas may comprise from 0.5% by mass oxygen to 99.9% by mass oxygen, such as from 1% by mass oxygen to 99.9% by mass oxygen, 10% by mass oxygen to 99.9% by mass oxygen, 20% by mass oxygen to 99.9% by mass oxygen, 50% by mass oxygen to 99.9% by mass oxygen, 0.5% by mass oxygen to 80% by mass oxygen, 0.5% by mass oxygen to 60% by mass oxygen, 0.5% by mass oxygen to 40% by mass oxygen, or 0.5% by mass oxygen to 30% by mass oxygen. In one or more embodiments, oxygen-containing gas may be air.

In one or more embodiments, the temperature of the deactivated catalyst may be from 550° C. to 800° C. For example, the temperature of the deactivated catalyst may be from 600° C. to 800° C., from 650° C. to 800° C., from 700° C. to 800° C., from 750° C. to 800° C., from 550° C. to 750° C., from 550° C. to 700° C., from 550° C. to 650° C., from 550° C. to 600° C., or from 600° C. to 650° C. In one or more embodiments, the temperature of the second portion of the reactivated catalyst may be from 700° C. to 900° C. For example, the temperature of the second portion of the reactivated catalyst may be from 750° C. to 900° C., from 800° C. to 900° C., from 850° C. to 900° C., from 700° C. to 850° C., from 700° C. to 800° C., from 700° C. to 750° C., or from 750° C. to 950° C. In one or more embodiments, the temperature of the deactivated catalyst increases when combined with the second portion of the reactivated catalyst. In some embodiments, the temperature of the deactivated catalyst may be from 600° C. to 850° C. after combining with the second portion of the reactivated catalyst. For example, the temperature of the deactivated catalyst may be from 650° C. to 850° C., from 700° C. to 850° C., from 750° C. to 850° C., from 800° C. to 850° C., from 600° C. to 800° C., from 600° C. to 750° C., from 600° C. to 700° C., from 600° C. to 750° C., from 650° C. to 750° C., or from 700° C. to 800° C. after combining with the second portion of the reactivated catalyst.

Still referring to FIG. 1, the mixed catalyst stream may enter combustor 350 where the mixed catalyst stream is then contacted with a second oxygen-containing gas stream. One or more of the first oxygen-containing gas stream and/or the second oxygen-containing gas stream may be air. The second oxygen-containing gas stream may enter combustor 350 via pipe 428. The second oxygen-containing gas stream may facilitate the combustion of one or more fuel gases or supplemental gases present in the combustor 350 and combust at least a portion of coke still present on the catalyst in the combustor 350. The catalyst is then passed out of the combustor 350 and through the riser 330 to a riser termination separator 378, where the gas and solid components from the riser 330 are at least partially separated. The vapor and remaining solids are transported to a secondary separation device 320 in the catalyst separation section 310 where the remaining catalyst is separated from the gases from the catalyst processing (e.g., gases emitted by combustion of spent catalyst or supplemental fuel, referred to herein as flue gas). The flue gas may pass out of the catalyst processing portion 300 via outlet pipe 432. A first portion of the separated catalyst (also referred to as a first portion of reactivated catalyst) is then passed through the oxygen treatment zone 370 within the catalyst separation section 310 to the upstream reactor section 250 via standpipe 424 and transport riser 430, where it is further utilized in a catalytic reaction. A second portion of the separated catalyst (also referred to as a second portion of reactivated catalyst) is passed towards the combustor 350 via standpipe 385 and combines with the deactivated catalyst from the reactor portion 200 to form the mixed catalyst stream that then enters the combustor 350. The second portion of catalyst may be exposed to oxygen containing gas for at least 5 seconds or even greater than 30 seconds (such as to a few minutes), which may be less than the time on oxygen for the first portion of catalyst. The catalyst, in operation, may cycle between the reactor portion 200 and the catalyst processing portion 300. In general, the processed chemical streams, including the feed streams and product streams may be gaseous, and the catalyst may be fluidized particulate solid.

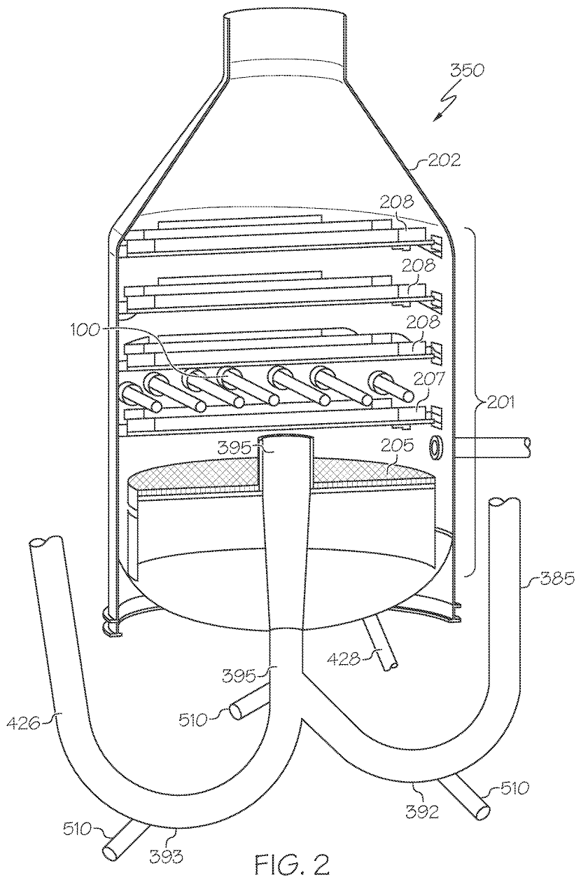

Referring to FIG. 2, a schematic cutaway view of an embodiment of a combustor 350 is shown. FIG. 2 shows a combustor 350 used as a fluidized fuel gas combustor system for a catalytic dehydrogenation process. However, as detailed herein, the chemical feed distributor 100 may be employed in a variety of vessels. Referring again to FIG. 2, the combustor 350 may include a lower portion 201 generally in the shape of a cylinder and an upper portion comprising a frustum 202. The angle between the frustum 202 and an internal horizontal imaginary line drawn at the intersection of the frustum 202 and the lower portion 201 may range from 10 to 80 degrees. All individual values and subranges from 10 to 80 degrees are included and disclosed herein; for example the angle between the tubular and frustum 202 components can range from a lower limit of 10, 40 or 60 degrees to an upper limit of 30, 50, 70 or 80 degrees. For example, the angle can be from 10 to 80 degrees, or in the alternative, from 30 to 60 degrees, or in the alternative, from 10 to 50 degrees, or in the alternative, from 40 to 80 degrees. Furthermore, in alternative embodiments, the angle can change along the height of the frustum 202, either continuously or discontinuously. In some embodiments, the combustor 350 may be, or may not be, lined with a refractory material.

The deactivated catalyst may pass towards the combustor 350 via standpipe 426 and the second portion of the reactivated catalyst may pass towards the combustor 350 via standpipe 385, where the deactivated catalyst and the second portion of the reactivated catalyst may combine and form a mixed catalyst stream in pipe 395. The mixed catalyst stream may pass upwards towards the air distributors 205. Above the air distributors 205 may be a grid 207. Above the grid 207 may be a plurality of chemical feed distributors 100. One or more additional grids 208 may be positioned within the combustor 350 above the chemical feed distributors 100. In embodiments, the chemical feed distributors 100 may enter the combustor 350 and traverse substantially across the combustor 350 as described in U.S. Publication No. US 2017/0087528.

Referring generally now to the catalyst processing portion 300, as depicted in FIG. 1, the combustor 350 of the catalyst processing portion 300 may be in fluid communication with the riser 330. The second oxygen-containing gas stream may be passed through pipe 428 into the combustor 350. The combustor 350 and riser 330, collectively referred to as the catalyst combustion reactor 302, may operate with similar or identical fluidization regimes as to what was disclosed with respect to the upstream reactor section 250 and downstream reactor section 230 of the reactor portion 200. That is, the combustor 350 may operate as a fluidized bed, such as in a fast fluidized, turbulent, or bubbling bed upflow reactor, while the riser 330 may operate in more of a plug flow manner, such as in a riser reactor. Geometries as described with respect to the upstream reactor section 250 and downstream reactor section 230 may equally apply to the combustor 350 and riser 330. Additionally, the combustor 350 may also include a fuel inlet 354, which may supply a fuel, such as a hydrocarbon stream, to the combustor 350.

In one or more embodiments, the first oxygen-containing gas stream and the second oxygen-containing gas stream may combine in the combustor 350 and have a combined oxygen-containing gas flow rate, wherein the flow rate of the first oxygen-containing gas stream is 1% to 15% of the combined oxygen-containing gas flow rate. It is to be understood that since the first oxygen-containing gas stream can pass the mixed catalyst stream to the combustor 350, the first-oxygen containing gas stream, after contacting and reacting with the mixed catalyst stream, will also be present in the combustor 350, thus contributing to the amount of oxygen-containing gas needed in the combustor 350. As such, the amount of the second oxygen-containing gas that will be needed during the combustion step in the combustor 350 will be less since the presence of the first-oxygen containing gas stream will supplemental the total amount of oxygen-containing gas present in the combustor 350. Importantly, this will reduce the costs associated with providing a larger amount of the second oxygen-containing gas stream to the combustor 350 that would be necessary if the mixed catalyst stream was not contacted with and passed to the combustor 350 by the first oxygen-containing gas stream.

As described in one or more embodiments, following separation of flue gas from catalyst in the riser termination separator 378 and secondary separation device 320, treatment of the processed catalyst with an oxygen-containing gas is conducted in the oxygen treatment zone 370. In some embodiments, the oxygen treatment zone 370 includes a fluid solids contacting device. The fluid solids contacting device may include baffles or grid structures to facilitate contact of the processed catalyst with the oxygen-containing gas. Examples of fluid solid contacting devices are described in further detail in U.S. Pat. Nos. 9,827,543 and 9,815,040. The fluidization regime within the oxygen treatment zone may be bubbling bed type fluidization. The oxygen treatment zone 370 may include an oxygen-containing gas inlet 372, which may supply an oxygen-containing gas to the oxygen treatment zone 370 for oxygen treatment of the catalyst.

In one or more embodiments, the light olefins may be present in a “product stream” sometimes called an “olefin-containing effluent” and include light olefins. Such a stream exits the reactor system of FIG. 1 and may be subsequently processed. As used in the present disclosure, the term “light olefins” refers to one or more of ethylene, propylene, and butene. The term butene includes any isomers of butene, such as α-butylene, cis-β-butylene, trans-β-butylene, and isobutylene. In some embodiments, the olefin-containing effluent includes at least 25 wt. % light olefins based on the total weight of the olefin-containing effluent. For example, the olefin-containing effluent may include at least 30 wt. % light olefins, at least 35 wt. % light olefins, at least 40 wt. % light olefins, at least 45 wt. % light olefins, at least 55 wt. % light olefins, or at least 60 wt. % light olefins based on the total weight of the olefin-containing effluent. The olefin-containing effluent may further comprise unreacted components of the feed stream, as well as other reaction products that are not considered light olefins. The light olefins may be separated from unreacted components in subsequent separation steps.

In non-limiting examples, the reactor system 102 described herein may be utilized to produce light olefins from hydrocarbon feed streams. Light olefins may be produced from a variety of hydrocarbon feed streams by utilizing different reaction mechanisms. For example, light olefins may be produced by at least dehydrogenation reactions, dehydrogenation reactions with selective hydrogen combustion, cracking reactions, dehydration reactions, and methanol-to-olefin reactions. These reaction types may utilize different feed streams and different particulate solids to produce light olefins. It should be understood that when “catalysts” are referred to herein, they may equally refer to the particulate solid referenced with respect to the system of FIG. 1.

According to one or more embodiments, the reaction may be a dehydrogenation reaction. According to such embodiments, the hydrocarbon feed stream may comprise one or more of ethyl benzene, ethane, propane, n-butane, and i-butane. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethyl benzene. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of ethane, propane, n-butane, and i-butane.

In one or more embodiments, the dehydrogenation reaction may utilize gallium and/or platinum particulate solids as a catalyst. In such embodiments, the particulate solids may comprise a gallium and/or platinum catalyst. As described herein, a gallium and/or platinum catalyst comprises gallium, platinum, or both. The gallium and/or platinum catalyst may be carried by an alumina or alumina silica support, and may optionally comprise potassium. Such gallium and/or platinum catalysts are disclosed in U.S. Pat. No. 8,669,406, which is incorporated herein by reference in its entirety. However, it should be understood that other suitable catalysts may be utilized to perform the dehydrogenation reaction.

In one or more embodiments, the reaction mechanism may be dehydrogenation followed by combustion (in the same chamber). In such embodiments, a dehydrogenation reaction may produce hydrogen as a byproduct, and an oxygen carrier material may contact the hydrogen and promote combustion of the hydrogen, forming water. Examples of such reaction mechanisms, which are contemplated as possible reactions mechanisms for the systems and methods described herein, are disclosed in WO 2020/046978, the teachings of which are incorporated by reference in their entirety herein.

According to one or more embodiments, the reaction may be a cracking reaction. According to such embodiments, the hydrocarbon feed stream may comprise one or more of naphtha, n-butane, or i-butane. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of naphtha. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of naphtha, n-butane, and i-butane.

In one or more embodiments, the cracking reaction may utilize one or more zeolites as a catalyst. In such embodiments, the particulate solids may comprise one or more zeolites. In some embodiments, the one or more zeolites utilized in the cracking reaction may comprise a ZSM-5 zeolite. However, it should be understood that other suitable catalysts may be utilized to perform the cracking reaction. For example, suitable catalysts that are commercially available may include Intercat Super Z Excel or Intercat Super Z Exceed. In additional embodiments, the cracking catalyst may comprise, in addition to a catalytically active material, platinum. For example, the cracking catalyst may include from 0.001 wt. % to 0.05 wt. % of platinum. The platinum may be sprayed on as platinum nitrate and calcined at an elevated temperature, such as around 700° C. Without being bound by theory, it is believed that the addition of platinum to the catalyst may allow for easier combustion of supplemental fuels, such as methane.

According to one or more embodiments, the reaction may be a dehydration reaction. According to such embodiments, the hydrocarbon feed stream may comprise one or more of ethanol, propanol, or butanol. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethanol. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propanol. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of butanol. In additional embodiments, the hydrocarbon feed stream or may comprise at least 50 wt. %, at least 60 wt. %, at least 70 wt. %, at least 80 wt. %, at least 90 wt. %, at least 95 wt. % or even at least 99 wt. % of the sum of ethanol, propanol, and butanol.

In one or more embodiments, the dehydration reaction may utilize one or more acid catalysts. In such embodiments, the particulate solids may comprise one or more acid catalysts. In some embodiments, the one or more acid catalysts utilized in the dehydration reaction may comprise a zeolite (such as ZSM-5 zeolite), alumina, amorphous aluminosilicate, acid clay, or combinations thereof. For example, commercially available alumina catalysts which may be suitable, according to one or more embodiments, include SynDol (available from Scientific Design Company), V200 (available from UOP), or P200 (available from Sasol). Commercially available zeolite catalysts which may be suitable include CBV 8014, CBV 28014 (each available from Zeolyst). Commercially available amorphous aluminosilicate catalysts which may be suitable include silica-alumina catalyst support, grade 135 (available from Sigma Aldrich). However, it should be understood that other suitable catalysts may be utilized to perform the dehydration reaction.

According to one or more embodiments, the reaction may be a methanol-to-olefin reaction. According to such embodiments, the hydrocarbon feed stream may comprise methanol. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of methanol.

In one or more embodiments, the methanol-to-olefin reaction may utilize one or more zeolites as a catalyst. In such embodiments, the particulate solids may comprise one or more zeolites. In some embodiments, the one or more zeolites utilized in the methanol-to-olefin reaction may comprise a one or more of a ZSM-5 zeolite or a SAPO-34 zeolite. However, it should be understood that other suitable catalysts may be utilized to perform the methanol-to-olefin reaction.

### EXAMPLES

Examples are provided herein. The Examples should not be viewed as limiting on the claimed embodiments hereinafter provided.

### Example 1

A reactor system including a reactor portion and a catalyst processing portion where the deactivated catalyst combines with a recycled portion of the reactivated catalyst from the catalyst processing portion ran for 60 cycles. Thus, the process of using the catalyst to conduct a dehydrogenation reaction in the reactor portion and combusting methane in the catalyst processing portion occurred 60 times. This trial is referred to as the inventive trial. Conversely, a reactor system including a reactor portion and a catalyst processing portion where the deactivated catalyst is directly sent to the combustor of the catalyst processing portion and does not combine with a recycle stream of reactivated catalyst from the catalyst processing portion ran for 60 cycles. Thus, the process of using the catalyst to conduct a dehydrogenation reaction in the reactor portion and combusting methane in the catalyst processing portion occurred 60 times. This trial is referred to as the comparative trial.

Table 1 below illustrates the percent conversion of methane that was achieved for the inventive trial and the comparative trial. As can be seen, the inventive trial that combined the deactivated catalyst with a portion of the reactivated catalyst from the catalyst processing portion achieved a higher percent conversion of the methane in the combustor at each cycle than the comparative trial that only sent the deactivated catalyst directly to the combustor.

TABLE 1
Methane Conversion versus Number of Cycles

| Cycle | Methane Conversion (%) | Methane Conversion (%) |
|---|---|---|
| Number | Inventive | Comparative |
| 1 | 83 | 79 |
| 5 | 79 | 76 |
| 10 | 76 | 71 |
| 15 | 75 | 69 |
| 20 | 74 | 68 |
| 25 | 73 | 68 |
| 30 | 72 | 67 |
| 35 | 72 | 67 |
| 40 | 71 | 66 |
| 45 | 70 | 66 |
| 50 | 68 | 65 |
| 55 | 68 | 65 |
| 60 | 68 | 64 |

### Example 2

This example illustrates the effectiveness of combining the deactivated catalyst with the recycled reactivated catalyst prior to passing this mixed catalyst stream to the combustor. The process conditions and pipe dimensions for the section of piping where the two catalyst streams mix are summarized in Table 2 below.

TABLE 2
Process Conditions and Dimensions of Catalyst Pre-Mixing Pipe

| | |
|---|---|
| Catalyst Density (Kg/m³) | 2000 |
| Deactivated Catalyst Flow Rate (Kg/hr) | 2,400,000 |
| Deactivated Catalyst Temperature (° C.) | 610 |
| Recycled Reactivated Catalyst Flow Rate | 2,400,000 |
| (Kg/hr) | |
| Recycled Reactivated Temperature (° C.) | 730 |
| First Oxygen-Containing Gas Flow Rate | 16,000 |
| (Kg/hr) | |
| Pipe Exit Pressure (bar-a) | 3 |
| Pipe Inside Diameter (m) | 1.473 |
| Pipe Length (m) | 7.620 |
| Gas Superficial Velocity (m/sec) | 2.3 |
| Catalyst Flux (Kg/m²-sec) | 782 |
| Coking Velocity (m/sec) | 5.7 |
| Flow Regime | Dense Phase Lift |

The mixing of the deactivated catalyst and recycled reactivated catalyst was simulated using a computational fluid dynamics (CFD) model developed in Ansys Fluent V19.2 using a drag model that has been extensively validated against experimental data. The predicted coefficient of variance (CoV) as shown in Table 3 was calculated using Equation 1 below, where X_{deactivated,i} represents the mass fraction of the deactivated catalyst over the mixed catalyst at location i of the pipe where the mixed catalyst stream enters the combustor.

$$COV = \frac{\sum \vert {X}_{deactivated , i} - {X}_{deactivated , mean} \vert}{{X}_{deactivated , mean}} \qquad ( Equation 1 )$$

TABLE 3
Predicted Mixing of Deactivated Catalyst and Recycled Reactivated
Catalyst along Pipe that Enters the Combustor

| Distance from The Pre-mixing Pipe | Coefficient of |
|---|---|
| Entrance (m) | Variance (%) |
| 6 | 0.3 |
| 4.7 | 2.9 |
| 3.4 | 4.9 |
| 2.2 | 6.8 |
| 1.1 | 21.0 |
| 0 | 25.7 |

Table 3 shows the mixing of the deactivated catalyst and recycled reactivated catalyst along the pipe length. Each percent of CoV corresponds to a 1.2° C. temperature difference between the catalyst streams. It can be seen that the model predicted at 2.5 m

$$( corresponding to \frac{3 {G}_{s} {D}^{2}}{{ρ}_{p}} )$$

above the pre-mixing pipe entrance, CoV is reduced to ~6.6% (~8° C. temperature variation). This demonstrates the effectiveness of this invention to combine the deactivated and recycled reactivated catalyst prior to the combustor, and ensures the mixed catalyst to uniformly achieve the target regeneration temperature.

The present disclosure includes one or more non-limiting aspects. A first aspect includes a method for forming light olefins in a reactor system, the method comprising: reacting a feed stream in the presence of a catalyst in a reactor to form a product stream and a deactivated catalyst comprising coke; separating at least a portion of the product stream from the deactivated catalyst; passing the deactivated catalyst to a combustor in a catalyst processing portion of the reactor system and processing the deactivated catalyst to produce a reactivated catalyst and a flue gas, wherein coke is removed from the deactivated catalyst in the combustor; separating the reactivated catalyst from the flue gas and separating the reactivated catalyst into a first portion and a second portion; passing the first portion of the reactivated catalyst to the reactor; combining the second portion of the reactivated catalyst with the deactivated catalyst upstream of the combustor to form a mixed catalyst stream, wherein the mixed catalyst stream is contacted with a first oxygen-containing gas stream upstream of the combustor and wherein coke on the deactivated catalyst is oxidized when contacted with the first oxygen-containing gas stream upstream of the combustor; and passing the mixed catalyst stream to the combustor and contacting the mixed catalyst stream with a second oxygen-containing gas stream while in the combustor, wherein the sum of the molar flow rate of the first oxygen-containing gas stream and the molar flow rate of the second oxygen-containing gas define a total oxygen-containing gas molar flow rate, and the molar flow rate of the first oxygen-containing gas stream is 1% to 15% of the total oxygen-containing gas molar flow rate.

A second aspect includes any above aspect, wherein a flow rate of the mixed catalyst stream entering the combustor is from 0.5 m/s to 4 m/s.

A third aspect includes any above aspect, wherein the second portion of the reactivated catalyst is passed in an oxygen-containing gas for greater than 5 seconds before combining with the deactivated catalyst.

A fourth aspect includes any above aspect, wherein the temperature of the deactivated catalyst increases when combined with the second portion of the reactivated catalyst.

A fifth aspect includes any above aspect, wherein the temperature of the deactivated catalyst is from 550° C. to 800° C. and the temperature of second portion of the reactivated catalyst is from 700° C. to 900° C.

A sixth aspect includes any above aspect, further comprising combusting a supplemental fuel in the combustor.

A seventh aspect includes any above aspect, wherein the supplemental fuel comprises hydrogen, methane, ethane, propane, or natural gas.

An eighth aspect includes any above aspect, wherein the mixed catalyst stream facilitates the combustion of the supplemental fuel.

A ninth aspect includes any above aspect, wherein the feed stream is reacted through a dehydrogenation reaction, a dehydrogenation reaction with selective hydrogen combustion, a cracking reaction, a dehydration reaction, or a methanol-to-olefin reaction.

A tenth aspect includes any above aspect, wherein the product stream comprises one or more of ethylene, propylene, styrene, or butene.

An eleventh aspect includes any above aspect, wherein the product stream comprises at least 25 wt. % light olefins.

A twelfth aspect includes any above aspect, wherein combining the second portion of the reactivated catalyst with the deactivated catalyst upstream of the combustor to form the mixed catalyst stream is done in a vessel or pipe operated in a dense phase lift fluidization regime.

A thirteenth aspect includes any above aspect, wherein the vessel or pipe is operated with a superficial gas velocity of from 0.3 m/s to 5 m/s and a solid flux of from 245 kg/m²-s to 1,710 kg/m²-s.

A fourteenth aspect includes any above aspect, wherein the vessel or pipe has a chocking velocity and the vessel or pipe is operated with a superficial gas velocity that is lower than the chocking velocity.

A fifteenth aspect includes any above aspect, wherein combining the second portion of the reactivated catalyst with the deactivated catalyst upstream of the combustor to form the mixed catalyst stream results in a temperature difference of less than or equal to 10° C. between the second portion of the reactivated catalyst and the deactivated catalyst prior to entering the combustor.

It will be apparent to those skilled in the art that various modifications and variations can be made to the presently disclosed technology without departing from the spirit and scope of the technology. Since modifications combinations, sub-combinations and variations of the disclosed embodiments incorporating the spirit and substance of the presently disclosed technology may occur to persons skilled in the art, the technology should be construed to include everything within the scope of the appended claims and their equivalents. Additionally, although some aspects of the present disclosure may be identified herein as preferred or particularly advantageous, it is contemplated that the present disclosure is not limited to these aspects.

It is noted that the various details described in this disclosure should not be taken to imply that these details relate to elements that are essential components of the various embodiments described in this disclosure, even in cases where a particular element is illustrated in each of the drawings that accompany the present description. Unless specifically identified as such, no feature disclosed and described herein should be construed as “essential”. Contemplated embodiments of the present technology include those that include some or all of the features of the appended claims.

For the purposes of describing and defining the present disclosure it is noted that the term “about” are utilized in this disclosure to represent the inherent degree of uncertainty that may be attributed to any quantitative comparison, value, measurement, or other representation. The term “about” are also utilized in this disclosure to represent the degree by which a quantitative representation may vary from a stated reference without resulting in a change in the basic function of the subject matter at issue.

In relevant cases, where a composition is described as “comprising” one or more elements, embodiments of that composition “consisting of” or “consisting essentially of” those one or more elements is contemplated herein.

It should be appreciated that compositional ranges of a chemical constituent in a stream or in a reactor should be appreciated as containing, in some embodiments, a mixture of isomers of that constituent. For example, a compositional range specifying butene may include a mixture of various isomers of butene. It should be appreciated that the examples supply compositional ranges for various streams, and that the total amount of isomers of a particular chemical composition can constitute a range.

It is noted that one or more of the following claims and the detailed description utilize the terms “where” or “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

As would be understood in the context of the term as used herein, the term “passing” may include directly passing a substance between two portions of the disclosed system and, in some other instances, to mean indirectly passing a substance between two portions of the disclosed system. For example, indirect passing may include steps where the named substance passes through an intermediate separation device, valve, sensor, etc.

It should be understood that any two quantitative values assigned to a property may constitute a range of that property, and all combinations of ranges formed from all stated quantitative values of a given property are contemplated in this disclosure. Where multiple ranges for a quantitative value are provided, these ranges may be combined to form a broader range, which is contemplated in the embodiments described herein.

1. A method for forming light olefins in a reactor system, the method comprising:
reacting a feed stream in the presence of a catalyst in a reactor to form a product stream and a deactivated catalyst comprising coke;
separating at least a portion of the product stream from the deactivated catalyst;
passing the deactivated catalyst to a combustor in a catalyst processing portion of the reactor system and processing the deactivated catalyst to produce a reactivated catalyst and a flue gas, wherein coke is removed from the deactivated catalyst in the combustor;
separating the reactivated catalyst from the flue gas and separating the reactivated catalyst into a first portion and a second portion;
passing the first portion of the reactivated catalyst to the reactor;
combining the second portion of the reactivated catalyst with the deactivated catalyst upstream of the combustor to form a mixed catalyst stream, wherein the mixed catalyst stream is contacted with a first oxygen-containing gas stream upstream of the combustor and wherein coke on the deactivated catalyst is oxidized when contacted with the first oxygen-containing gas stream upstream of the combustor; and
passing the mixed catalyst stream to the combustor and contacting the mixed catalyst stream with a second oxygen-containing gas stream while in the combustor;
wherein the sum of the molar flow rate of the first oxygen-containing gas stream and the molar flow rate of the second oxygen-containing gas define a total oxygen-containing gas molar flow rate, and the molar flow rate of the first oxygen-containing gas stream is 1% to 15% of the total oxygen-containing gas molar flow rate.

2. The method of claim 1, wherein a flow rate of the mixed catalyst stream entering the combustor is from 0.5 m/s to 4 m/s.

3. The method of claim 1, wherein the second portion of the reactivated catalyst is passed in an oxygen-containing gas for greater than 5 seconds before combining with the deactivated catalyst.

4. The method of claim 1, wherein the temperature of the deactivated catalyst increases when combined with the second portion of the reactivated catalyst.

5. The method of claim 1, wherein the temperature of the deactivated catalyst is from 550° C. to 800° C. and the temperature of second portion of the reactivated catalyst is from 700° C. to 900° C.

6. The method of claim 1, further comprising combusting a supplemental fuel in the combustor.

7. The method of claim 6, wherein the supplemental fuel comprises hydrogen, methane, ethane, propane, or natural gas.

8. The method of claim 6, wherein the mixed catalyst stream facilitates the combustion of the supplemental fuel.

9. The method of claim 1, wherein the feed stream is reacted through a dehydrogenation reaction, a dehydrogenation reaction with selective hydrogen combustion, a cracking reaction, a dehydration reaction, or a methanol-to-olefin reaction.

10. The method of claim 1, wherein the product stream comprises one or more of ethylene, propylene, styrene, or butene.

11. The method of claim 1, wherein the product stream comprises at least 25 wt. % light olefins.

12. The method of claim 1, wherein combining the second portion of the reactivated catalyst with the deactivated catalyst upstream of the combustor to form the mixed catalyst stream is done in a vessel or pipe operated in a dense phase lift fluidization regime.

13. The method of claim 12, wherein the vessel or pipe is operated with a superficial gas velocity of from 0.3 m/s to 5 m/s and a solid flux of from 245 kg/m²-s to 1,710 kg/m²-s.

14. The method of claim 12, wherein the vessel or pipe has a choking velocity and the vessel or pipe is operated with a superficial gas velocity that is lower than the chocking velocity.

15. The method of claim 1, wherein combining the second portion of the reactivated catalyst with the deactivated catalyst upstream of the combustor to form the mixed catalyst stream results in a temperature difference of less than or equal to 10° C. between the second portion of the reactivated catalyst and the deactivated catalyst prior to entering the combustor.
